(19) United States
(12) Patent Application Publication
(10) Pub. No.: US 2004/0214789 A1
(43) Pub. Date: Oct. 28, 2004

(54) Therapeutics

(76) Inventors: Barry V.L. Potter, Bath (GB); Andreas H. Guse, Hamburg (DE); Georg W. Mayr, Pinneberg (DE); Katrin Schweitzer, Magedeburg (DE)

Correspondence Address: ST. ONGE STEWARD JOHNSTON & REENS, LLC, 986 BEDFORD STREET, STAMFORD, CT, 06905-5619, US

(21) Appl. No.: 10/730,589

(22) Filed: Dec. 8, 2003

Related U.S. Application Data
(63) Continuation of application No. PCT/GB02/02695, filed on Jun. 6, 2002.

(30) Foreign Application Priority Data
Jun. 7, 2001 (GB) 0113923.7

(51) Int. Cl. A61K 31/7076; A61K 31/52

(52) U.S. Cl. 514/45; 514/256; 514/263.2; 514/263.24

(57) ABSTRACT

The use of a compound of formula (Ia):

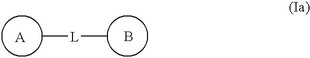

wherein A and B are independently selected from a cyclic ring, wherein each of which cyclic rings A and B may be optionally substituted at at least one ring position; and

L is a suitable linker;

or a pharmaceutically acceptable salt thereof, in the manufacture of a medicament for use in inhibiting ADP-ribosyl cyclase.

This application is a continuation of pending International Patent Application No. PCT/GB02/02695 filed Jun. 6, 2002 which designates the United States and claims priority of pending British Application No. 0113923.7 filed Jun. 7, 2001.

### FIELD OF THE INVENTION

The present invention relates to therapeutics. In particular, but not exclusively, the present invention relates to the modulation of T cell activity via a cyclic ADP ribose mediated pathway or via a nicotinic acid adenine dinucleotide phosphate (NAADP+) mediated pathway by the inhibition of ADP-ribosyl cyclase. The invention also relates to compounds capable of modulating the activity of T cells by the inhibition of ADP-ribosyl cyclase. The invention also relates to treating diseases using such compounds and methods for identifying such compounds.

### BACKGROUND TO THE INVENTION

Adaptive or specific immune responses are normally stimulated when an individual is exposed to a foreign antigen. Specific immunity is mediated by lymphocytes, e.g. B and T lymphocytes. During an immune response, recognition of an antigen leads to activation of lymphocytes that specifically recognise that particular antigen. The lymphocytes proliferate and differentiate into specialised effector cells. The immune response culminates in the development of mechanisms that ultimately eliminate the antigen.

Adaptive immune responses are critical components of host defence during protection against foreign antigens, such as infectious organisms or toxins. However, specific immune responses are also sometimes elicited by antigens not associated with infectious agents, and this may cause serious disease. For example, one of the most remarkable properties of specific immunity is the ability to distinguish between self antigens and foreign antigens. Thus, the lymphocytes in each individual are able to recognise and respond to numerous foreign antigens but are normally unresponsive to potentially antigenic substances present in the individual itself. Unresponsiveness to self antigens is an acquired process that has to be learned by the individual's lymphocytes and has to be maintained throughout life.

Abnormalities in the induction or maintenance of self-tolerance lead to immune responses against self antigens, and debilitating diseases that are commonly called autoimmune diseases. The spectrum of autoimmune disorders ranges from organ specific diseases (such as thyroiditis, insulitis, multiple sclerosis, iridocyclitis, uveitis, orchitis, hepatitis, Addison's disease, myasthenia gravis) to systemic illnesses such as rheumatoid arthritis or lupus erythematosus.

Another example in which specific immunity against antigens that are not associated with infections causes severe medical problems are rejections of transplanted allografts. In fact, adaptive immune responses to grafted tissues are the major impediment to successful transplantation in most cases.

It is not known what causes the breakdown of tolerance and the initiation of an autoimmune response. However, the mechanisms of tissue destruction in autoimmune diseases and in allograft rejection are essentially the same as those operating in protective immunity. It is generally believed that both autoimmune reactions and allograft rejections are initiated and perpetuated by a response involving T cells. Thus, in the absence of a specific therapy for any of the autoimmune diseases or for allograft rejection, many therapeutic strategies currently employed aim at down modulating the activity of the immune system, in particular by reducing or preventing the activation of T cells.

Recently, monoclonal antibodies to T cell surface antigens, that inhibit T cell activation, or substances that interfere with intracellular T cell activation pathways, such as Cyclosporin A or FK506, have been introduced for the treatment of both allograft rejection and several autoimmune diseases. However, current approaches for the treatment of undesirable T cell activation have been associated with a number of side effects related to general immunosuppression and therefore cannot be considered to be optimal therapy.

Stimulation of T-lymphocytes via the T cell receptor/CD3 complex (TCR/CD3) is a critical step in T cell activation and subsequent clonal expansion. Previous studies have shown that activation of the TCR/CD3-complex involves the elevation of the free cytosolic Ca²⁺ concentration ([Ca²⁺]i) by at least two mechanisms, a rapid elevation caused by Ca²⁺ release from intracellular stores mediated by inositol (1,4,5) trisphosphate (Ins(1,4,5)P₃), and a prolonged elevation that is completely dependent on the influx of extracellular calcium (reviewed in Guse, 1998). Ca²⁺-release is activated by the calcium mobilizing second messengers Ins(1,4,5)P₃ (Jayaraman et al, 1995) and cADPR (Guse et al., 1999). Recent work indicates that Ins(1,4,5)P₃ primarily acts during the initial phase of Ca²⁺ -signaling in T cells, whereas cADPR is essentially involved in the sustained phase of Ca²⁺ -signaling.

The exact mechanism of Ca²⁺ signalling in T cells is still unclear, but it is of fundamental importance for proliferation and clonal expansion, and thus for a functional immune response. An improved understanding of the signalling pathways involved in T cell activation may be of assistance in developing strategies to stimulate a desirable adaptive immune response or to suppress inappropriate T cell activity.

In WO00/37089 it was shown that the potent Ca²⁺ mobilising compound cyclic ADP-ribose (cADPR), which is found in a variety of eukaryotic cells, is essentially required for sustained Ca²⁺ signalling mediated via stimulation of the T cell receptor/CD3 (TCR/CD3) complex. Thus, it was shown that cADPR has a role as a second messenger in T cell activation via the TCR/CD3 complex.

The stimulation of the TCR/CD3 complex resulted in activation of soluble ADP-ribosyl cyclase and a sustained elevation of the intracellular level of cADPR. A causal relationship between elevated cADPR, sustained Ca²⁺ signalling and activation of T cells was revealed by inhibition of TCR/CD3-stimulated Ca²⁺ signalling, cell proliferation and expression of early and late activation marker, CD25 and HLA-DR, using the membrane-permanent antagonist 7-deaza-8-Br-cADPR (WO98/43992). Expression of the molecular target for cADPR in T cells, and type 3 ryanodine receptor/Ca²⁺ channel, was demonstrated both by RT-PCR and immunoprecipitation/western blotting. Increased cADPR significantly and specifically stimulated the apparent association of [³H] ryanodine indicating a direct modulatory effect on channel opening. Thus, the causal relation and biological significance of the major constituents of the cADPR/Ca²⁺ signalling pathway have been demonstrated in human T cells (Guse et al 1999).

The UK patent application number 0019234.4 (unpublished) shows that NAADP+specifically and dose-dependently stimulates Ca²⁺ signalling in human T cells. At an activating concentration, NAADP+ either evokes repetitive and long-lasting Ca²⁺ oscillations or a single Ca²⁺-spike of high amplitude. The actual mechanism of formation of NAADP+ in vivo is unknown, but it is suggested in UK patent application number 0019234.4 that at least three enzyme activities have been implicated. It is intimated that ADP-ribosyl cyclase may play a role, since it is known to be able to make NAADP+ in vitro from NADP+ by base exchange.

The NAADP+ can be self-inactivating. An inactivating concentration of NAADP+ inhibits subsequent stimulation of Ca²⁺ signaling via the T cell receptor/CD3. For example, inactivation of the NAADP+/Ca²⁺-release system almost completely abolishes subsequent Ins(1,4,5)P₃— or cADPR-mediated Ca⁺-signaling. This shows that a functional NAADP+/Ca²⁺ release system is essential for T lymphocyte Ca²⁺ signaling.

### SUMMARY OF THE INVENTION

The present inventors have now shown that human T lymphocytes produce an ADP-ribosyl cyclase intracellularly. The present inventors have further shown a class of compounds which completely or substantially completely inhibit an ADP-ribosyl cyclase. In particular, the class of compounds may completely or substantially completely inhibit a soluble ADP-ribosyl cyclase and/or an intracellular ADP-ribosyl cyclase. The class of compounds may completely or substantially completely inhibit a membrane bound intracellular ADP-ribosyl cyclase. By completely or substantially completely inhibiting an ADP-ribosyl cyclase, the concentration of cADPR and/or NAADP+ may be modulated. Thus, the compounds may be capable of modulating T cell activity, by regulation of the cADPR/Ca²⁺ signaling pathway and/or by regulation of the NAADP+/Ca²⁺ signaling pathway. In this way, there is provided an important means of controlling T cell responses in a variety of T cell mediated immune disorders.

In a broad aspect the present invention relates to the use of a compound of formula (Ia):

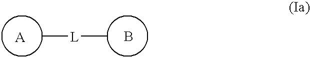

wherein A and B are independently selected from a cyclic ring, wherein each of which cyclic rings A and B may be optionally substituted at at least one ring position; and

L is a suitable linker;

or a pharmaceutically acceptable salt thereof, in the manufacture of a medicament for use in inhibiting ADP-ribosyl cyclase.

One aspect of the present invention relates to the use of a compound of formula (Ia) wherein one or more of the cyclic rings A and B is a heterocyclic ring.

Another aspect of the present invention relates to the use of a compound of formula (Ia) wherein one or more of the cyclic rings A and B is a five membered ring.

In a preferred aspect the present invention relates to the use of a compound of formula (Ia) wherein the cyclic ring A has the formula (II):

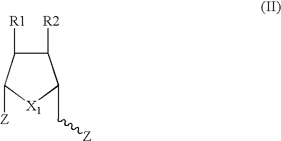

wherein X₁ is independently selected from O, S, CH₂ or a halo derivative thereof;

each of R₁ or R₂ is a substituent group independently selected from OH, OR, SH, SR, halo (preferably F), NH₂, NHR or

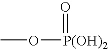

and wherein R is independently a hydrocarbyl group, preferably C₁₋₁₂, preferably C₁₋₆, alkyl or acyl group (which may be optionally substituted), and

Z is a hydrocarbyl.

Preferably, when X₁ is selected from a halo derivative of CH₂ the halo derivative is CF₂.

Preferably, X₁ is O.

Preferably, each of R₁ or R₂ is an OH.

In a preferred aspect the present invention relates to the use of a compound of formula (Ia) wherein the cyclic ring B has the formula (III):

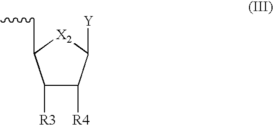

wherein X₂ is independently selected from O, S, CH₂ or a halo derivative thereof;

each of R₃ or R₄ is a substituent group independently selected from OH, OR, SH, SR, halo (preferably F), NH₂, NHR or

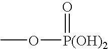

and wherein R is independently a hydrocarbyl group, preferably a C₁₋₁₂, preferably C₁₋₆, alkyl or acyl group (which may be optionally substituted); and

Y is a hydrocarbyl.

Preferably, X₂ is O.

Preferably, each of R₃ or R₄ is an OH.

Suitably, each of Y or Z may be independently selected from an aromatic group or a substituted aromatic group.

Advantageously, each of Y or Z may be independently selected from a heteroaromatic group or a substituted heteroaromatic group.

Preferably, the heteroaromatic group or the substituted heteroaromatic group comprises a purine or a substituted purine structure.

Preferably, Z is a pyridine or a substituted pyridine.

Preferably, Y is a purine or a substituted purine.

In a preferred aspect of the present invention Z has the formula (IV):

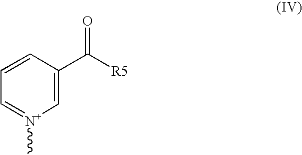

wherein R₅ is NH₂, OH or

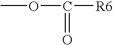

wherein R₆ is a hydrocarbyl group, preferably C₁₋₁₂, preferably C₁₋₆, alkyl or aryl group (which may be optionally substituted).

In a preferred aspect of the present invention Y comprises two fused heterocyclic rings, wherein each of said heterocyclic rings independently comprises nitrogen and carbon atoms in their respective rings, and wherein each of said heterocyclic rings may be optionally substituted at at least one ring position.

Preferably, Y has the formula (V):

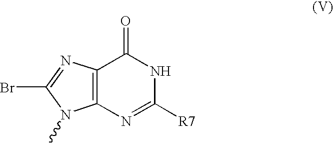

wherein R₇ is independently H or NH₂.

Another aspect of the present invention relates to the use of a compound of formula (Ia) wherein the linker is non-hydrolysable.

In a preferred aspect the present invention relates to the use of a compound of formula (Ia) wherein the linker has the formula (VI):

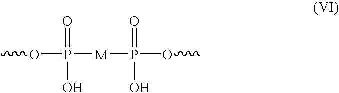

wherein M is independently selected from O, NH, CH₂ or a halo derivative thereof.

Preferably, when M is selected from a halo derivative of CH₂ the halo derivative is CF₂.

Suitably, the linker may be selected from any one or more of the group comprising: a phosphate, a polyphosphate, a phosphorothioate, a polyethylene glycol, an alkyl, an alkylaryl, a peptide and a polyamine.

In a preferred aspect the present invention relates to the use of a compound of formulae (Ib):

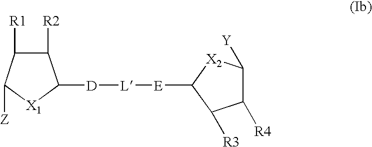

wherein D and E are independently selected from O, S, CH₂ or a halo derivative thereof; wherein each of X₁ and X₂ is independently selected from O, S, CH₂ or a halo derivative thereof;

each of R₁, R₂, R₃ or R₄ is a substituent group independently selected from OH, OR, SH, SR, halo (preferably F), NH₂, NHR or

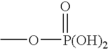

and wherein R is independently a hydrocarbyl group, preferably a C₁₋₁₂, preferably C₁₋₆, alkyl or acyl group (which may be optionally substituted);

each of Z and Y is a hydrocarbyl; and

L′ is the remainder of linker L;

or a pharmaceutically acceptable salt thereof.

Preferably, when D or E are independently selected from a halo derivative of CH₂ the halo derivative is CF₂.

In another preferred aspect the present invention relates to the use of a compound which is a nicotinamide adenine dinucleotide analogue or a nicotinic acid adenine dinucleotide phosphate analogue.

In a further preferred aspect the present invention relates to the use of one or more of the following compounds: nicotinamide. 8-bromohypoxanthine dinucleotide; 7-deaza-nicotinamide hypoxanthine dinucleotide; nicotinamide hypoxanthine dinucleotide; 6-thio-nicotinamide hypoxanthine dinucleotide; nicotinamide 8-bromoguanine dinucleotide.

In a preferred aspect the present invention relates to the use of a compound of the present invention for use in the preparation of a medicament for use in modulating the immune response of a mammal.

In a further preferred aspect the present invention relates to the use of a compound of the present invention for use in the preparation of a medicament for use in treating an autoimmune disease or a graft rejection.

Preferably, the medicament is for use in treating an autoimmune disease selected from thyroiditis, insulitis, multiple sclerosis, iridocyclitis, uveitis, orchitis, hepatitis, Addison's disease, myasthenia gravis, rheumatoid arthritis and lupus erythematosus.

The present invention further relates to the use of a compound of the present invention for use in the preparation of a medicament for use in treating or preventing an immune disorder in a human or animal.

The present invention further provides a pharmaceutical composition comprising a compound according to the present invention or a pharmaceutically acceptable salt thereof admixed with a pharmaceutically acceptable carrier, diluent or excipient.

The pharmaceutical composition may comprise one or more additional pharmaceutically active compounds.

The present invention also provides a compound of formula (Ib):

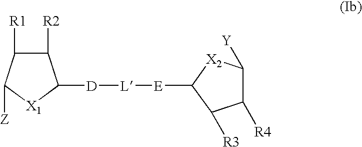

wherein D and E are independently selected from O, S, CH₂ or a halo derivative thereof;

wherein each of X₁ and X₂ is independently selected from O, S, CH₂ or a halo derivative thereof;

each of R₁, R₂, R₃ or R₄ is a substituent group independently selected from OH, OR, SH, SR, halo (preferably F), NH₂, NHR or

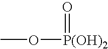

and wherein R is independently a hydrocarbyl group, preferably a C₁₋₁₂, preferably C₁₋₆, alkyl or acyl group (which may be optionally substituted);

each of Z and Y is a hydrocarbyl; and

L′ is the remainder of linker L.

Preferably, when D or E are independently selected from a halo derivative of CH₂ the halo derivative is CF₂.

In a preferred aspect the present invention relates to a compound of formula (Ib) wherein each of X₁ and X₂ is O.

In a further preferred aspect the present invention relates to a compound of formula (Ib) wherein each of R₁, R₂, R₃ or R₄ is an OH.

Another aspect of the present invention relates to a compound of formula (Ib) wherein Y or Z is independently selected from an aromatic group or a substituted aromatic group

Another aspect of the present invention relates to a compound of formula (Ib) wherein each of Y or Z is independently selected from a heteroaromatic group or a substituted heteroaromatic group.

Suitably, the heteroaromatic group or the substituted heteroaromatic group may comprise a purine or a substituted purine structure.

Preferably, Z is a pyridine or a substituted pyridine.

Preferably, Y is a purine or a substituted purine.

In a preferred aspect the present invention relates to a compound of formula (Ib) wherein Z has the formula (IV):

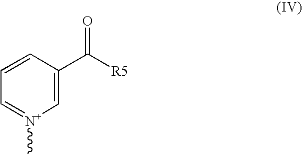

wherein R₅ is NH₂, OH or

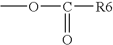

wherein R₆ is a hydrocarbyl group, preferably C₁₋₁₂, preferably C₁₋₆, alkyl or aryl group (which may be optionally substituted).

In a further aspect the present invention relates to a compound of formula (Ib) wherein Y comprises two fused heterocyclic rings, wherein each of said heterocyclic rings independently comprises nitrogen and carbon atoms in their respective rings, and wherein each of said heterocyclic rings may be optionally substituted at at least one ring position.

In a preferred aspect the present invention relates to a compound of formula (Ib) wherein Y has the formula (V):

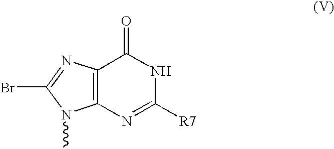

wherein R₇ is independently H or NH₂.

In one aspect the present invention relates to a compound of formula (Ib) wherein the linker is non-hydrolysable.

In a preferred aspect the present invention relates to a compound of formula (Ib) wherein the linker has the formula (VI):

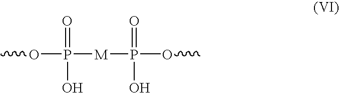

wherein M is independently selected from O, NH, CH₂ or a halo derivative thereof.

Preferably, when M is selected from a halo derivative of CH₂ the halo derivative is CF₂.

Another aspect of the present invention relates to a compound of formula (Ib) wherein the compound is a nicotinamide adenine dinucleotide analogue or a nicotinic acid adenine dinucleotide phosphate analogue.

Compounds of formula (Ib) which are particularly suitable for use in the present invention include nicotinamide 8-bromohypoxanthine dinucleotide; 7-deaza-nicotinamide hypoxanthine dinucleotide; nicotinamide hypoxanthine dinucleotide; 6-thio-nicotinamide hypoxanthine dinucleotide; nicotinamide 8-bromoguanine dinucleotide.

The present invention further provides a compound of the present invention for use as a medicament.

The present invention also provides the use of a compound of the present invention for use in the manufacture of a medicament for use in inhibiting ADP-ribosyl cyclase.

The present invention also relates to a medicament comprising a compound of the present invention

The present invention further provides a method of inhibiting ADP-ribosyl cyclase comprising the step of contacting an ADP-ribosyl cyclase with a compound or composition of the present invention.

The present invention further provides a method of modulating the concentration of cADPR and/or NAADP+ in a cell comprising the step of contacting an ADP-ribosyl cyclase with a compound or composition of the present invention.

Preferably, the concentration of cADPR is decreased. Preferably, the concentration of NAADP+ is either decreased to a concentration below an activating concentration, for example to less than or equal to about 10 nM or increased up to an inactivating concentration, for example greater than or equal to 10 μM.

The present invention also provides a method of modulating intracellular Ca²⁺ levels in a T-cell comprising the step of contacting an ADP-ribosyl cyclase with a compound or a composition of the present invention.

Preferably, the intracellular Ca²⁺ levels in a T cell are reduced to a level below that normally required to stimulate T cells.

The present invention further provides a method of modulating T cell activity, which comprises the step of contacting an ADP-ribosyl cyclase with a compound or a composition of the present invention.

Preferably, T cell activity is decreased. However, T cell activity may, in some instances, be increased.

The step of contacting an ADP-ribosyl cyclase with a compound or a composition of the present invention may be carried out in vitro.

Alternatively, the step of contacting an ADP-ribosyl cyclase with a compound or a composition of the present invention may be carried out in vivo.

The present invention further provides a method of treating or preventing a disease in a human or animal patient which method comprises administering to the patient an effective amount of a compound or a composition of the present invention.

The present invention further provides a pharmaceutical pack comprising one or more compartments, wherein at least one compartment comprises one or more of the compounds or a composition of the present invention.

The invention yet further provides a pharmaceutical pack for use in the treatment of autoimmune disease or graft rejection; the pack comprising one or more compartments; wherein at least one of said compartments houses one or more compounds of the present invention. In the pack of the present invention, the compound may be admixed with a pharmaceutically acceptable carrier, diluent or excipient. In addition, or in the alternative, the pack of the present invention may comprise a further compartment which houses a pharmaceutically acceptable carrier, diluent or excipient.

Suitably, at least one of the compartments houses one or more compounds, and at least one of the other compartments of the pack comprises one or more other pharmaceutically active agents.

Where the pack of the present invention comprises more than one compound, the compounds may be in different forms. Likewise, where the pack of the invention comprises one or more compounds together with one or more other pharmaceutically active agents, the one or more compounds and the other pharmaceutically active agents may be in different forms. By way of example, one may be a solution or tablet and the other may be a cream. In one preferred embodiment of the present invention, one component of the pack is to be applied topically and the other component of the pack is to be applied systemically. It is to be understood that the pack could contain extra compartments.

The present invention yet further provides a process of preparation of a pharmaceutical composition of the present invention, said process comprising admixing one or more of the compounds of the present invention with a pharmaceutically acceptable diluent, excipient or carrier.

The present invention further provides an assay method for identifying an agent that can directly or indirectly inhibit ADP-ribosyl cyclase in order to treat an autoimmune disease or a graft rejection, the assay method comprising: contacting an agent with ADP-ribosyl cyclase; and measuring the activity of ADP-ribosyl cyclase; wherein a downregulation of activity of ADP-ribosyl cyclase in the presence of the agent is indicative that the agent may be useful in the treatment of an autoimmune disease or a graft rejection.

The invention further relates to a process comprising the steps of:

(a) performing the above-mentioned assay,

(b) identifying one or more agents that can directly or indirectly downregulate the activity of ADP-ribosyl cyclase; and

(c) preparing a quantity of those one or more identified agents.

In addition, another aspect of the invention relates to a method of treating an autoimmune disease or graft rejection, by downregulating the activity of ADP-ribosyl cyclase in vivo with an agent; wherein the agent is capable of directly or indirectly downregulating the activity of ADP-ribosyl cyclase in an in vitro assay method; wherein the in vitro assay method is as described hereinbefore.

Yet another aspect of the invention relates to the use of an agent in the preparation of a pharmaceutical composition for the treatment of an autoimmune response or a graft rejection, wherein the agent is capable of directly or indirectly downregulating the activity of ADP-ribosyl cyclase when assayed in vitro by the assay method described above.

The invention also relates to an agent identified by the assay method described above.

Preferably, the agent identified by the assay method described above is for use in medicine.

Even more preferably, the agent identified by the assay method described above is for use in treating an autoimmune disease or a graft rejection.

Another aspect of the invention relates to the use of one or more compounds according to the present invention in an assay for identifying candidate compounds that are capable of influencing the activity of ADP-ribosyl cyclase.

The agents identified by any such assay method may be used as therapeutic agents—i.e. in therapy applications.

Preferably, the compound of the present invention is used in modulating T cell activity.

Suitably, the compound may be used in inducing T cell anergy.

Advantageously, the compound is used in blocking T cell proliferation and/or differentiation. This is particularly the case when the compound is used to provide a very low (non-active) concentration of NAADP+ (≦10 nM) or is used to provide an inactivating concentration of NAADP+ (≧10 μM) and/or is used to decrease the concentration of cADPR.

Suitably, the compound may be used to stimulate T cell proliferation and/or differentiation. This is particularly the case when the compound is used to provide an activating concentration of NAADP+.

It is known that the intracellular concentration of NAADP+ dictates whether or not it is activating (i.e. stimulates a rise in intracellular Ca²⁺ levels) or inactivating (i.e. inhibits TCR/CD3-associated Ca²⁺ signalling). An intracellular concentration of 10 nM NAADP+ evokes repetitive and long-lasting Ca²⁺ oscillations of low amplitude, while 50 and 100 nM produces a rapid and high initial Ca²⁺ peak followed by trains of smaller Ca²⁺ oscillations. Higher concentrations of NAADP+ (1 and 10 μM) gradually reduce the initial Ca²⁺ peak. Thus an “activating concentration” of NAADP+ may be between 5 nM and 1 μM, preferably between 5 and 100 nM. An intracellular concentration of 100 μM NAADP+ causes complete self-inactivation of Ca²⁺-signals. Thus an “inactivating concentration” of NAADP+ may be greater than 10 μM, preferably 100 μM or more.

Compounds of the invention which inhibit T cell proliferation and/or differentiation, or induce T cell anergy may be used in treating diseases characterised by an excessive or inappropriate T cell response, such as autoimmune diseases, allergies and allograft rejection. Candidate autoimmune diseases include thyroiditis, insulitis, multiple sclerosis, iridocyclitis, uveitis, orchitis, hepatitis, Addison's disease, myasthenia gravis, rheumatoid arthritis and lupus erythematosus.

Compounds of the invention which induce or enhance T cell proliferation and/or differentiation or prevent the induction of T cell anergy may be used generally to boost or induce T cell immune responses. Virtually all adaptive immune responses require the activation of T cells and their differentiation into cytokine-producing cells. Thus these compounds may be used generally to prevent and treat conditions such as infectious diseases (such as viral or bacterial infections), cancers and, in particular, immunodeficiencies characterised by impaired T cell function (such as AIDS).

The term “compound” is intended to encompass isomeric forms (such as stereoisomers and/or geometric and/or optical isomers, and mixtures thereof), chemical derivatives, mimetics, solvates and salts of the compounds.

As used herein, the term “hydrocarbyl” refers to a group comprising at least C and H that may optionally comprise one or more other suitable substituents. Examples of such substituents may include halo-, alkoxy-, nitro-, an alkyl group, or a cyclic group. In addition to the possibility of the substituents being a cyclic group, a combination of substituents may form a cyclic group. If the hydrocarbyl group comprises more than one C then those carbons need not necessarily be linked to each other. For example, at least two of the carbons may be linked via a suitable element or group. Thus, the hydrocarbyl group may contain heteroatoms. Suitable heteroatoms will be apparent to those skilled in the art and include, for instance, sulphur, nitrogen, oxygen, silicon and phosphorus.

For some embodiments, preferably the hydrocarbyl group is alkyl, alkoxy, alkenyl, alkylene, acyl and alkenylene groups—which may be unbranched- or branched-chain.

For some embodiments, preferably the hydrocarbyl group is C₁₋₁₂ alkyl, C₁₋₁₂ alkoxy, C₁₋₁₂ alkenyl, C₁₋₁₂ alkylene, C₁₋₁₂ acyl, and C₁₋₁₂ alkenylene groups—which may be unbranched- or branched-chain.

For some embodiments, preferably the hydrocarbyl group is C₁₋₆ alkyl, C₁₋₆ alkoxy, C₁₋₆ alkenyl, C₁₋₆ alkylene, C₁₋₆ acyl, and C₁₋₆ alkenylene groups—which may be unbranched- or branched-chain.

It is to be appreciated that all references herein to treatment include one or more of curative, palliative and prophylactic treatment. Preferably, the term treatment includes at least curative treatment and/or palliative treatment.

Stereo and Geometric Isomers

Some of the compounds of the present invention may exist as stereoisomers and/or geometric isomers—e.g. they may possess one or more asymmetric and/or geometric centres and so may exist in two or more stereoisomeric and/or geometric forms. The present invention contemplates the use of all the individual stereoisomers and geometric isomers of those compounds, and mixtures thereof. The terms used in the claims encompass these forms, provided said forms retain the appropriate functional activity (though not necessarily to the same degree).

Solvates

The present invention also includes the use of solvate forms of the compound of the present invention. The terms used in the claims encompass these forms.

Pro-Drug

As indicated, the present invention also includes the use of pro-drug forms of the compounds of the present invention. The terms used in the claims encompass these forms. Examples of prodrugs include entities that have certain protected group(s) and which may not possess pharmacological activity as such, but may, in certain instances, be administered (such as orally or parenterally) and thereafter metabolised in the body to form the compounds of the present invention which are pharmacologically active.

It will be further appreciated that certain moieties known as “pro-moieties”, for example as described in “Design of Prodrugs” by H. Bundgaard, Elsevier, 1985 (the disclosure of which is hereby incorporated by reference), may be placed on appropriate functionalities of the compounds. Such prodrugs are also included within the scope of the invention.

Mimetic

In one embodiment of the present invention, the compound may be a mimetic. As used herein, the term “mimetic” relates to any chemical which includes, but is not limited to, a peptide, polypeptide, antibody or other organic chemical which has the same qualitative activity or effect as a reference agent.

Chemical Derivative

In one embodiment of the present invention, the compound may be a derivative. The term “derivative” as used herein includes chemical modification of a compound. Illustrative of such chemical modifications would be replacement of hydrogen by a halo group, an alkyl group, an acyl group or an amino group.

Chemical Modification

In one embodiment of the present invention, the compound may be a chemically modified compound.

The chemical modification of a compound of the present invention may either enhance or reduce hydrogen bonding interaction, charge interaction, hydrophobic interaction, van der Waals interaction or dipole interaction between the agent and the target.

General Assay Techniques

In one aspect, the identified compounds according to the present invention may act as a model (for example, a template) for the development of other compounds. The compounds employed in such a test may be free in solution, affixed to a solid support, borne on a cell surface, or located intracellularly. The abolition of activity or the formation of binding complexes between the compound and the agent being tested may be measured.

The assay of the present invention may be a screen, whereby a number of agents are tested. In one aspect, the assay method of the present invention is a high through put screen.

Techniques for drug screening may be based on the method described in Geysen, European Patent Application 84/03564, published on Sep. 13, 1984. In summary, large numbers of different small peptide test compounds are synthesised on a solid substrate, such as plastic pins or some other surface. The peptide test compounds are reacted with a suitable compound or fragment thereof and washed. Bound entities are then detected —such as by appropriately adapting methods well known in the art. A purified compound can also be coated directly onto plates for use in a drug screening techniques. Alternatively, non-neutralising antibodies can be used to capture the peptide and immobilise it on a solid support.

This invention also contemplates the use of competitive drug screening assays in which neutralising antibodies capable of binding a compound according to the present invention specifically compete with a test compound for binding to a compound according to the present invention.

Another technique for screening provides for high throughput screening (HTS) of agents having suitable binding affinity to the substances and is based upon the method described in detail in WO 84/03564.

It is expected that the assay methods of the present invention will be suitable for both small and large-scale screening of test compounds as well as in quantitative assays.

Reporters

A wide variety of reporters may be used in the assay methods (as well as screens) of the present invention with preferred reporters providing conveniently detectable signals (e.g. by spectroscopy). By way of example, a number of companies such as Pharmacia Biotech (Piscataway, N.J.), Promega (Madison, Wis.), and US Biochemical Corp (Cleveland, Ohio) supply commercial kits and protocols for assay procedures. Suitable reporter molecules or labels include those radionuclides, enzymes, fluorescent, chemiluminescent, or chromogenic agents as well as substrates, cofactors, inhibitors, magnetic particles and the like. Patents teaching the use of such labels include U.S. Pat. No. 3,817,837; U.S. Pat. No. 3,850,752; U.S. Pat. No. 3,939,350; U.S. Pat. No. 3,996,345; U.S. Pat. No. 4,277,437; U.S. Pat. No. 4,275,149 and U.S. Pat. No. 4,366,241.

Pharmaceutical Salts

The compounds of the present invention may be administered as pharmaceutically acceptable salts. Typically, a pharmaceutically acceptable salt may be readily prepared by using a desired acid or base, as appropriate. The salt may precipitate from solution and be collected by filtration or may be recovered by evaporation of the solvent.

Pharmaceutically-acceptable salts are well known to those skilled in the art, and for example include those mentioned by Berge et al, in J. Pharm. Sci. 66, 1-19 (1977). Suitable acid addition salts are formed from acids which form non-toxic salts and include the hydrochloride, hydrobromide, hydroiodide, nitrate, sulphate, bisulphate, phosphate, hydrogenphosphate, acetate, trifluoroacetate, gluconate, lactate, salicylate, citrate, tartrate, ascorbate, succinate, maleate, fumarate, gluconate, formate, benzoate, methanesulphonate, ethanesulphonate, benzenesulphonate and p-toluenesulphonate salts.

When one or more acidic moieties are present, suitable pharmaceutically acceptable base addition salts can be formed from bases which form non-toxic salts and include the aluminium, calcium, lithium, magnesium, potassium, sodium, zinc, and pharmaceutically-active amines such as diethanolamine, salts.

The compounds of the present invention may exist in polymorphic form.

In addition, the compounds of the present invention may contain one or more asymmetric carbon atoms and therefore exists in two or more stereoisomeric forms. Where a compound contains an alkenyl or alkenylene group, cis (E) and trans (Z) isomerism may also occur. The present invention includes the individual stereoisomers of the compound and, where appropriate, the individual tautomeric forms thereof, together with mixtures thereof.

Separation of diastereoisomers or cis and trans isomers may be achieved by conventional techniques, e.g. by fractional crystallisation, chromatography or H.P.L.C. of a stereoisomeric mixture of the agent or a suitable salt or derivative thereof. An individual enantiomer of the compound may also be prepared from a corresponding optically pure intermediate or by resolution, such as by H.P.L.C. of the corresponding racemate using a suitable chiral support or by fractional crystallisation of the diastereoisomeric salts formed by reaction of the corresponding racemate with a suitable optically active acid or base, as appropriate.

The present invention also includes all suitable isotopic variations of the compound or a pharmaceutically acceptable salt thereof. An isotopic variation of a compound of the present invention or a pharmaceutically acceptable salt thereof is defined as one in which at least one atom is replaced by an atom having the same atomic number but an atomic mass different from the atomic mass usually found in nature. Examples of isotopes that can be incorporated into the compound and pharmaceutically acceptable salts thereof include isotopes of hydrogen, carbon, nitrogen, oxygen, phosphorus, sulphur, fluorine and chlorine Such as ²H, ³H, ¹³C, ¹⁴C, ¹⁵N, ¹⁷O, ¹⁸O, ³¹P, ³²P, ³⁵S, ¹⁸F and ³⁶Cl, respectively. Certain isotopic variations of the compound and pharmaceutically acceptable salts thereof, for example, those in which a radioactive isotope such as ³H or ¹⁴C is incorporated, are useful in drug and/or substrate tissue distribution studies. Tritiated, i.e., ³H, and carbon-14, i.e., ¹⁴C, isotopes are particularly preferred for their ease of preparation and detectability. Further, substitution with isotopes such as deuterium, i.e., ²H, may afford certain therapeutic advantages resulting from greater metabolic stability, for example, increased in vivo half-life or reduced dosage requirements and hence may be preferred in some circumstances. Isotopic variations of the compound of the present invention and pharmaceutically acceptable salts thereof of this invention can generally be prepared by conventional procedures using appropriate isotopic variations of suitable reagents.

The present invention also includes (wherever appropriate) the use of zwitterionic forms of the compounds of the present invention.

The terms used in the claims encompass one or more of the forms just mentioned.

Formulation

The component(s) of the present invention may be formulated into a pharmaceutical composition, such as by mixing with one or more of a suitable carrier, diluent or excipient, by using techniques that are known in the art.

Pharmaceutical Compositions

The present invention provides a pharmaceutical composition comprising a therapeutically effective amount of one or more compounds of the present invention and a pharmaceutically acceptable carrier, diluent or excipient (including combinations thereof).

The pharmaceutical compositions may be for human or animal usage in human and veterinary medicine and will typically comprise any one or more of a pharmaceutically acceptable diluent, carrier, or excipient. Acceptable carriers or diluents for therapeutic use are well known in the pharmaceutical art, and are described, for example, in Remington's Pharmaceutical Sciences, Mack Publishing Co. (A. R. Gennaro edit. 1985). The choice of pharmaceutical carrier, excipient or diluent can be selected with regard to the intended route of administration and standard pharmaceutical practice. The pharmaceutical compositions may comprise as—or in addition to—the carrier, excipient or diluent any suitable binder(s), lubricant(s), suspending agent(s), coating agent(s), solubilising agent(s).

Examples of suitable carriers include lactose, starch, glucose, methyl cellulose, magnesium stearate, mannitol, sorbitol and the like. Examples of suitable diluents include ethanol, glycerol and water.

Examples of suitable binders include starch, gelatin, natural sugars such as glucose, anhydrous lactose, free-flow lactose, beta-lactose, corn sweeteners, natural and synthetic gums, such as acacia, tragacanth or sodium alginate, carboxymethyl cellulose and polyethylene glycol.

Examples of suitable lubricants include sodium oleate, sodium stearate, magnesium stearate, sodium benzoate, sodium acetate, sodium chloride and the like.

Preservatives, stabilizers, dyes and even flavoring agents may be provided in the pharmaceutical composition. Examples of preservatives include sodium benzoate, sorbic acid and esters of p-hydroxybenzoic acid. Antioxidants and suspending agents may be also used.

There may be different composition/formulation requirements dependent on the different delivery systems. By way of example, the pharmaceutical composition of the present invention may be formulated to be administered using a mini-pump or by a mucosal route, for example, as a nasal spray or aerosol for inhalation or ingestable solution, or parenterally in which the composition is formulated by an injectable form, for delivery, by, for example, an intravenous, intramuscular or subcutaneous route. Alternatively, the formulation may be designed to be administered by a number of routes.

Where the composition is to be administered mucosally through the gastrointestinal mucosa, it should be able to remain stable during transit though the gastrointestinal tract; for example, it should be resistant to proteolytic degradation, stable at acid pH and resistant to the detergent effects of bile.

Where appropriate, the pharmaceutical compositions can be administered by inhalation, in the form of a suppository or pessary, topically in the form of a lotion, solution, cream, ointment or dusting powder, by use of a skin patch, orally in the form of tablets containing excipients such as starch or lactose, or in capsules or ovules either alone or in admixture with excipients, or in the form of elixirs, solutions or suspensions containing flavouring or colouring agents, or they can be injected parenterally, for example intravenously, intramuscularly or subcutaneously. For parenteral administration, the compositions may be best used in the form of a sterile aqueous solution which may contain other substances, for example enough salts or monosaccharides to make the solution isotonic with blood. For buccal or sublingual administration the compositions may be administered in the form of tablets or lozenges which can be formulated in a conventional manner.

For some embodiments, one or more compounds may also be used in combination with a cyclodextrin. Cyclodextrins are known to form inclusion and non-inclusion complexes with drug molecules. Formation of a drug-cyclodextrin complex may modify the solubility, dissolution rate, bioavailability and/or stability property of a drug molecule.

Drug-cyclodextrin complexes are generally useful for most dosage forms and administration routes. As an alternative to direct complexation with the drug the cyclodextrin may be used as an auxiliary additive, e.g. as a carrier, diluent or solubiliser. Alpha-, beta- and gamma-cyclodextrins are most commonly used and suitable examples are described in WO-A-91/11172, WO-A-94/02518 and WO-A-98/55148.

The pharmaceutical composition may comprise one or more additional pharmaceutically active compounds.

Chemical Synthesis Methods

The compounds of the present invention may be available commercially.

Alternatively, the compound of the invention may be prepared by chemical synthesis techniques.

It will be apparent to those skilled in the art that sensitive functional groups may need to be protected and deprotected during synthesis of a compound of the invention. This may be achieved by conventional techniques, for example as described in “Protective Groups in Organic Synthesis” by T W Greene and P G M Wuts, John Wiley and Sons Inc. (1991), and by P. J. Kocienski, in “Protecting Groups”, Georg Thieme Verlag (1994).

It is possible during some of the reactions that any stereocentres present could, under certain conditions, be epimerised, for example if a base is used in a reaction with a substrate having an having an optical centre comprising a base-sensitive group. It should be possible to circumvent potential problems such as this by choice of reaction sequence, conditions, reagents, protection/deprotection regimes, etc. as is well-known in the art.

The compounds and salts of the invention may be separated and purified by conventional methods.

Synthesis of Compounds Capable of Modulating ADP-Ribosyl Cyclase Activity

General Methods for the Synthesis of NAD⁺Analogues:

Phosphorylation of nucleosides. Nucleosides and their analogues (0.10 mmoles) dried in vacuo over P₂O₅ were dissolved in hot triethylphosphate under N₂. After cooling the mixture to 0° C., phosphorus oxychloride (3 equivalents) was added under N₂. The reaction was stirred at 0° C. for 3 h and warmed up to room temperature. After 24 h, the reaction was quenched by addition of ice-cold aqueous pyridine (3:1, v/v, pyridine:water) at 0° C. The solvents were removed under reduced pressure and the residue purified by anion exchange column chromatography using Fast Flow Q-Sepharose gel (50 ml) and a gradient of triethylammonium bicarbonate (TEAB) buffer (900 mL, 0-500 mM). Fractions containing the required material (>95% HPLC purity) were combined and concentrated. Excess TEAB was removed by coevaporating with isopropanol. The phosphorylated species were quantified using the Ames assay. Yields ranged from 40-80%. The monophosphorylated species were then dried in vacuo by co-evaporating with DMF.

Synthesis of pyrophosphate nucleotides. To a 2 ml solution of aqueous pyridine (3:1,v/v, pyridine:water) containing 2 equivalents of β-nicotinamide mononucleotide was added the required phosphorylated nucleoside. A large excess of dicyclohexylcarbodiimide (2 g) was then added and the heterogeneous mixture stirred for 5 days at room temperature under N₂. Water was added and the solution was stirred for 2 h. Dicyclohexylurea was then extracted with ether, while the newly formed pyrophosphate remained in the aqueous layer. Pyridine and water were removed under reduced pressure at 20° C. To simplify the purification of symmetrical dinucleotides, where no β-nicotinamide mononucleotide was used, the residue obtained after concentration was dissolved in 5 ml of 50 mM diethanolamine solution buffered at pH 9.8 containing 0.5 mM MgCl₂. One unit of alkaline phosphatase was added. The reaction was incubated at 17° C. for 30 min and followed by HPLC. The reaction was terminated by diluting the reaction solution with 100 mL of water. The solution was then loaded on a Q-Sepharose anion exchange chromatography column and the dinucleotide purified using similar conditions as described previously. Non-symmetrical dinucleotides were directly purified by Q-Sepharose anion exchange chromatography under similar conditions as described previously. Yields ranged from 10-80% NHD, 7-deaza-NHD and 6-thio-NHD, for which data are presented herein were synthesised in a similar way to that described above.

Therapy

As with the term “treatment”, the term “therapy” includes curative effects, alleviation effects, and prophylactic effects.

The therapy may be on humans or animals.

Therapeutic Uses

The compounds of the present invention may be used in therapy. In particular such compounds may be used to modulate T cell responses in vivo. Alternatively, T cells may be removed from a patient, treated with a compound of the present invention, and then returned to the patient (ex vivo therapy).

Compounds capable of blocking T cell proliferation and/or differentiation may be used against any disorder which is susceptible to prevention or treatment by the induction of an adaptive immune response. In particular, these compounds may be used to treat immunodeficiency disorders mechanistically related to a defect in T cell activation.

Examples of such disorders include a group commonly called autoimmune diseases. The spectrum of autoimmune disorders ranges from organ specific diseases (such as thyroiditis, insulitis, multiple sclerosis, iridocyclitis, uveitis, orchitis, hepatitis, Addison's disease, myasthenia gravis) to systemic illnesses such as rheumatoid arthritis or lupus erythematosus. Other disorders include immune hyperreactivity, such as allergic reactions.

In more detail: Organ-specific autoimmune diseases include multiple sclerosis, insulin dependent diabetes mellitus, several forms of anaemia (aplastic, haemolytic), autoimmune hepatitis, thyroiditis, insulitis, iridocyclitis, skleritis, uveitis, orchitis, myasthenia gravis, idiopathic thrombocytopenic purpura, inflammatory bowel diseases (Crohn's disease, ulcerative colitis).

Systemic autoimmune diseases include: rheumatoid arthritis, juvenile arthritis, scleroderma and systemic sclerosis, sjogren's syndrom, undifferentiated connective tissue syndrom, antiphospholipid syndrom, different forms of vasculitis (polyarteritis nodosa, allergic granulomatosis and angiitis, Wegner's granulomatosis, Kawasaki disease, hypersensitivity vasculitis, Henoch-Schoenlein purpura, Behcet's Syndrome, Takayasu arteritis, Giant cell arteritis, Thrombangiitis obliterans), lupus erythematosus, polymyalgia rheumatica, essentiell (mixed) cryoglobulinemia, Psoriasis vulgaris and psoriatic arthritis, diffus fasciitis with or without eosinophilia, polymyositis and other idiopathic inflammatory myopathies, relapsing panniculitis, relapsing polychondritis, lymphomatoid granulomatosis, erythema nodosum, ankylosing spondylitis, Reiter's syndrom, different forms of inflammatory dermatitis.

A more extensive list of disorders is given in WO-A-98/09985. For ease of reference, part of that list is now provided: unwanted immune reactions and inflammation including arthritis, including rheumatoid arthritis, inflammation associated with hypersensitivity, allergic reactions, asthma, systemic lupus erythematosus, collagen diseases and other autoimmune diseases, inflammation associated with atherosclerosis, arteriosclerosis, atherosclerotic heart disease, reperfusion injury, cardiac arrest, myocardial infarction, vascular inflammatory disorders, respiratory distress syndrome or other cardiopulmonary diseases, inflammation associated with peptic ulcer, ulcerative colitis and other diseases of the gastrointestinal tract, hepatic fibrosis, liver cirrhosis or other hepatic diseases, thyroiditis or other glandular diseases, glomerulonephritis or other renal and urologic diseases, otitis or other oto-rhino-laryngological diseases, dermatitis or other dermal diseases, periodontal diseases or other dental diseases, orchitis or epididimo-orchitis, infertility, orchidal trauma or other immune-related testicular diseases, placental dysfunction, placental insufficiency, habitual abortion, eclampsia, pre-eclampsia and other immune and/or inflammatory-related gynaecological diseases, posterior uveitis, intermediate uveitis, anterior uveitis, conjunctivitis, chorioretinitis, uveoretinitis, optic neuritis, intraocular inflammation, e.g. retinitis or cystoid macular oedema, sympathetic ophthalmia, scleritis, retinitis pigmentosa, immune and inflammatory components of degenerative fondus disease, inflammatory components of ocular trauma, ocular inflammation caused by infection, proliferative vitreo-retinopathies, acute ischaemic optic neuropathy, excessive scarring, e.g. following glaucoma filtration operation, immune and/or inflammation reaction against ocular implants and other immune and inflammatory-related ophthalmic diseases, inflammation associated with autoimmune diseases or conditions or disorders where, both in the central nervous system (CNS) or in any other organ, immune and/or inflammation suppression would be beneficial, Parkinson's disease, complication and/or side effects from treatment of Parkinson's disease, AIDS-related dementia complex HIV-related encephalopathy, Devic's disease, Sydenham chorea, Alzheimer's disease and other degenerative diseases, conditions or disorders of the CNS, inflammatory components of stokes, post-polio syndrome, immune and inflammatory components of psychiatric disorders, myelitis, encephalitis, subacute sclerosing pan-encephalitis, encephalomyelitis, acute neuropathy, subacute neuropathy, chronic neuropathy, Guillaim-Barre syndrome, Sydenham chora, myasthenia gravis, pseudo-tumour cerebri, Down's Syndrome, Huntington's disease, amyotrophic lateral sclerosis, inflammatory components of CNS compression or CNS trauma or infections of the CNS, inflammatory components of muscular atrophies and dystrophies, and immune and inflammatory related diseases, conditions or disorders of the central and peripheral nervous systems, post-traumatic inflammation, septic shock, infectious diseases, inflammatory complications or side effects of surgery or organ, inflammatory and/or immune complications and side effects of gene therapy, e.g. due to infection with a viral carrier, or inflammation associated with AIDS, to suppress or inhibit a humoral and/or cellular immune response, to treat or ameliorate monocyte or leukocyte proliferative diseases, e.g. leukaemia, by reducing the amount of monocytes or lymphocytes, for the prevention and/or treatment of graft rejection in cases of transplantation of natural or artificial cells, tissue and organs such as cornea, bone marrow, organs, lenses, pacemakers, natural or artificial skin tissue.

Compounds capable of stimulating (i.e. inducing or enhancing) T cell proliferation and/or differentiation or of preventing the induction of T cell anergy may be used generally to boost or induce T cell immune responses. Virtually all adaptive immune responses require the activation of T cells and their differentiation into cytokine-producing cells. Thus these compounds may be used generally to prevent and treat conditions such as infectious diseases (such as viral or bacterial infections), cancers and, in particular, immunodeficiencies characterised by impaired T cell function (such as AIDS).

Administration

The components of the present invention may be administered alone but will generally be administered as a pharmaceutical composition—e.g. when the components are is in admixture with a suitable pharmaceutical excipient, diluent or carrier selected with regard to the intended route of administration and standard pharmaceutical practice.

For example, the composition can be administered (e.g. orally or topically) in the form of tablets, capsules, ovules, elixirs, solutions or suspensions, which may contain flavouring or colouring agents, for immediate-, delayed-, modified-, sustained-, pulsed- or controlled-release applications.

If the pharmaceutical composition is a tablet, then the tablet may contain excipients such as microcrystalline cellulose, lactose, sodium citrate, calcium carbonate, dibasic calcium phosphate and glycine, disintegrants such as starch (preferably corn, potato or tapioca starch), sodium starch glycollate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia. Additionally, lubricating agents such as magnesium stearate, stearic acid, glyceryl behenate and talc may be included.

Solid compositions of a similar type may also be employed as fillers in gelatin capsules. Preferred excipients in this regard include lactose, starch, cellulose, milk sugar or high molecular weight polyethylene glycols. For aqueous suspensions and/or elixirs, the compound may be combined with various sweetening or flavouring agents, colouring matter or dyes, with emulsifying and/or suspending agents and with diluents such as water, ethanol, propylene glycol and glycerin, and combinations thereof.

The routes for administration (delivery) include, but are not limited to, one or more of: oral (e.g. as a tablet, capsule, or as an ingestable solution), topical, mucosal (e.g. as a nasal spray or aerosol for inhalation), nasal, parenteral (e.g. by an injectable form), gastrointestinal, intraspinal, intraperitoneal, intramuscular, intravenous, intrauterine, intraocular, intradermal, intracranial, intratracheal, intravaginal, intracerebroventricular, intracerebral, subcutaneous, ophthalmic (including intravitreal or intracameral), transdermal, rectal, buccal, vaginal, epidural, sublingual.

Where the composition comprises more than one compound, it is to be understood that not all of the components of the pharmaceutical need be administered by the same route.

Likewise, if the composition comprises more than one active component, then those components may be administered by different routes.

If a component of the present invention is administered parenterally, then examples of such administration include one or more of: intravenously, intra-arterially, intraperitoneally, intrathecally, intraventricularly, intraurethrally, intrasternally, intracranially, intramuscularly or subcutaneously administering the component; and/or by using infusion techniques.

For parenteral administration, the component is best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art.

As indicated, the component(s) of the present invention can be administered intranasally or by inhalation and is conveniently delivered in the form of a dry powder inhaler or an aerosol spray presentation from a pressurised container, pump, spray or nebuliser with the use of a suitable propellant, e.g. dichlorodifluoromethane, trichlorofluoromethane, dichlorotetrafluoroethane, a hydrofluoroalkane such as 1,1,1,2-tetrafluoroethane (HFA 134A™) or 1,1,1,2,3,3,3-heptafluoropropane (HFA 227EA™), carbon dioxide or other suitable gas. In the case of a pressurised aerosol, the dosage unit may be determined by providing a valve to deliver a metered amount. The pressurised container, pump, spray or nebuliser may contain a solution or suspension of the active compound, e.g. using a mixture of ethanol and the propellant as the solvent, which may additionally contain a lubricant, e.g. sorbitan trioleate. Capsules and cartridges (made, for example, from gelatin) for use in an inhaler or insufflator may be formulated to contain a powder mix of the agent and a suitable powder base such as lactose or starch.

Alternatively, the component(s) of the present invention can be administered in the form of a suppository or pessary, or it may be applied topically in the form of a gel, hydrogel, lotion, solution, cream, ointment or dusting powder. The component(s) of the present invention may also be dermally or transdermally administered, for example, by the use of a skin patch. They may also be administered by the pulmonary or rectal routes. They may also be administered by the ocular route. For ophthalmic use, the compounds can be formulated as micronised suspensions in isotonic, pH adjusted, sterile saline, or, preferably, as solutions in isotonic, pH adjusted, sterile saline, optionally in combination with a preservative such as a benzylalkonium chloride. Alternatively, they may be formulated in an ointment such as petrolatum.

For application topically to the skin, the component(s) of the present invention can be formulated as a suitable ointment containing the active compound suspended or dissolved in, for example, a mixture with one or more of the following: mineral oil, liquid petrolatum, white petrolatum, propylene glycol, polyoxyethylene polyoxypropylene compound, emulsifying wax and water. Alternatively, it can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyldodecanol, benzyl alcohol and water.

In a preferred embodiment of the invention, the pharmaceutical composition is administered orally.

T cells treated ex vivo are typically administered to the patient by intramuscular, intraperitoneal or intravenous injection, or by direct injection into the lymph nodes of the patient, preferably by direct injection into the lymph nodes. Typically from 10⁴ to 10⁸ treated cells, preferably from 10⁵ to 10⁷ cells, more preferably about 10⁶ cells are administered to the patient.

Dose Levels

Typically, a physician will determine the actual dosage which will be most suitable for an individual subject. The specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy.

The routes of administration and dosages described are intended only as a guide since a skilled practitioner will be able to determine readily the optimum route of administration and dosage for any particular patient depending on, for example, the age, weight and condition of the patient.

### EXAMPLES

The present invention will now be described only by way of example.

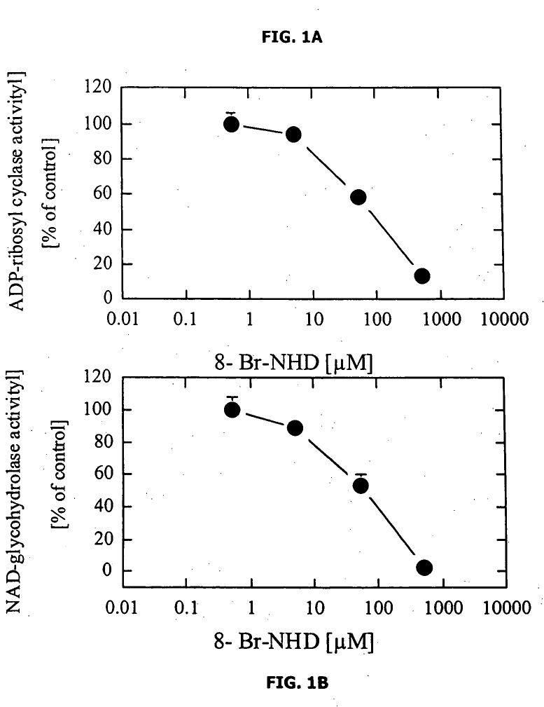

FIG. 1-a, b—Inhibition of partially purified, soluble ADP-ribosyl cyclase by 8-Br-NHD

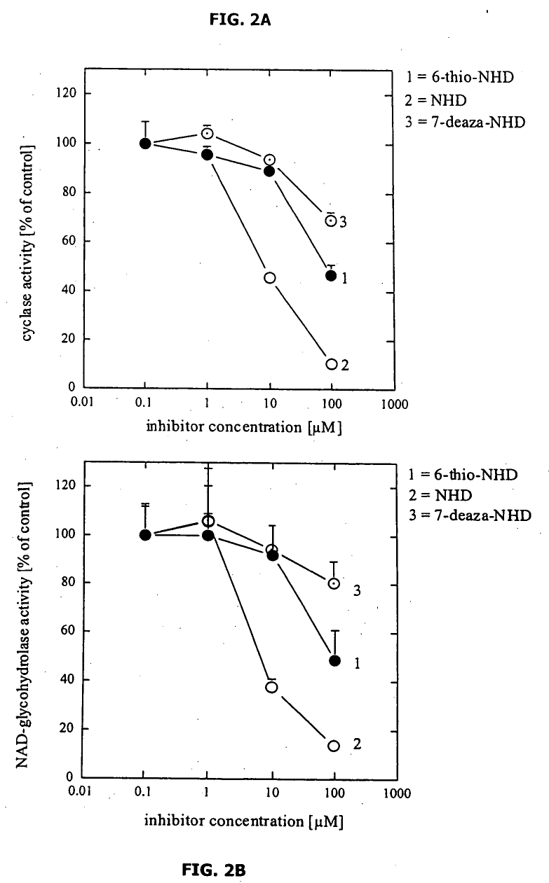

FIG. 2-a, b—Inhibition of intracellular, soluble ADP-ribosyl cyclase by various NHD analogues

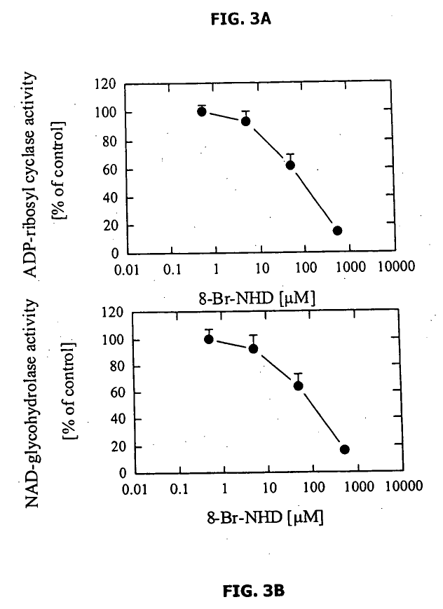

FIG. 3-a, b—Inhibition of intracellular, membrane-bound (P10) ADP-ribosyl cyclase by 8-Br-NHD

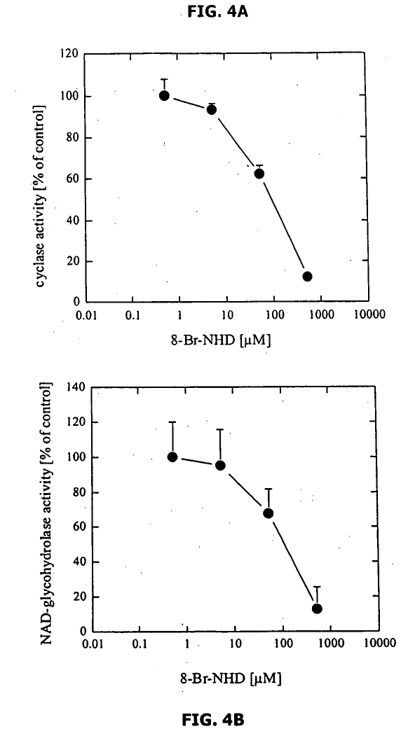

FIG. 4-a, b—Inhibition of cell surface, membrane-bound ADP-ribosyl cyclase by 8-Br-NHD

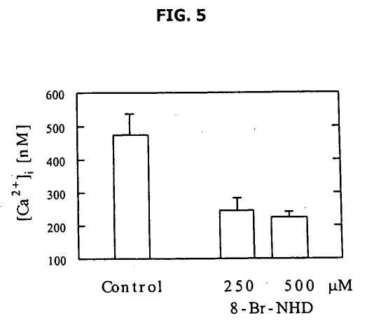

FIG. 5—Inhibition of TCR/CD3-mediated Ca²⁺-signalling of the human Jurkat T cell line by 8-Br-NHD

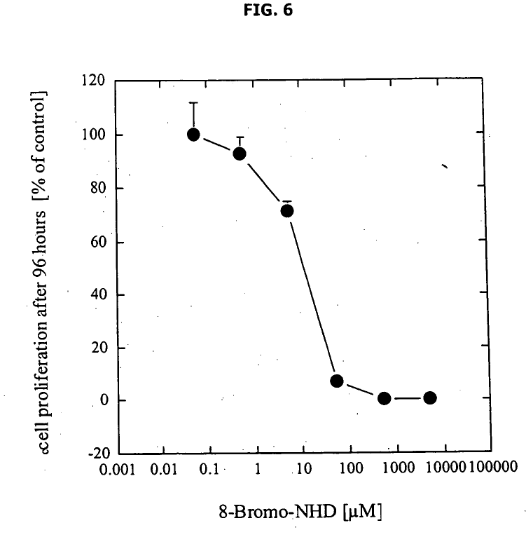

FIG. 6—Inhibition of proliferation of the human Jurkat T cell line by 8-Br-NHD

### DETAILED DESCRIPTION OF THE FIGURES

FIG. 1-a, b—Inhibition of intracellular, soluble ADP-ribosyl cyclase by 8-Br-NHD. Soluble protein (S100 protein) was prepared from Jurkat T cells (10⁹ cells) as described (Guse et al., Nature 1999) and soluble ADP-ribosyl cyclase was partially purified by hydroxyapatite chromatography. Partially purified ADP-ribosyl cyclase was then incubated with 100 μM substrate (1,N⁶-etheno-NAD) in the presence or absence of various concentrations of 8-Br-NHD. The formation of 1,N⁶-etheno-cADPR and 1,N⁶-etheno-ADPR was continuously monitored by fluorimetry (exc. 300 nm, em. 410 nm). Finally, the precise amount of products 1,N⁶-etheno-cADPR and 1,N⁶-etheno-ADPR was quantified by HPLC (da Silva et al., 1998a) using a fluorescence detector. Data are presented as mean ±SD (n≧3).

FIG. 2-a, b—Inhibition of intracellular, soluble ADP-ribosyl cyclase by various NHD analogues.

Soluble protein (S100 protein) was prepared from Jurkat T cells (10⁹ cells) as described (Guse et al., Nature 1999) and soluble ADP-ribosyl cyclase was partially purified by hydroxyapatite chromatography. Partially purified ADP-ribosyl cyclase was then incubated with 100 μM substrate (1,N⁶-etheno-NAD) in the presence or absence of various concentrations of 7-deaza-NHD, NHD and 6-thio-NHD. The formation of 1,N⁶-etheno-cADPR and 1,N⁶-etheno-ADPR was continuously monitored by fluorimetry (exc. 300 nm, em. 410 nm). Finally, the precise amount of products 1,N⁶-etheno-cADPR and 1,N⁶-etheno-ADPR was quantified by HPLC (da Silva et al., 1998a) using a fluorescence detector. Data are presented as mean±SD (n≧3).

FIG. 3—Inhibition of intracellular, membrane-bound (P10) ADP-ribosyl cyclase by 8-Br-NHD.

Heavy membrane (P10 membranes) were prepared from Jurkat T cells (109 cells) as described (Guse et al., Nature 1999). P10-ADP-ribosyl cyclase was then incubated with 100 μM substrate (1,N⁶-etheno-NAD) in the presence or absence of various concentrations of 8-Br-NHD. The formation of 1,N⁶-etheno-cADPR and 1,N⁶-etheno-ADPR was continuously monitored by fluorimetry (exc. 300 nm, em. 410 nm). Finally, the precise amount of products 1,N⁶-etheno-cADPR and 1,N⁶-etheno-ADPR was quantified by HPLC (da Silva et al., 1998a) using a fluorescence detector. Data are presented as mean±SD (n≧3).

FIG. 4—Inhibition of cell surface ADP-ribosyl cyclase by 8-Br-NHD.

Intact Jurkat T cells expressing ecto-ADP-ribosyl cyclase (da Silva et al., 1998b) were incubated with 100 μM substrate (1,N⁶-etheno-NAD) in the presence or absence of various concentrations of 8-Br-NHD. It is likely that this enzyme is CD38. The formation of 1,N⁶-etheno-cADPR and 1,N⁶-etheno-ADPR was continuously monitored by fluorimetry (exc. 300 nm, em. 410 nm). Finally, the precise amount of products 1,N⁶-etheno-cADPR and 1,N⁶-etheno-ADPR was quantified by HPLC (da Silva et al., 1998a) using a fluorescence detector. Data are presented as mean±SD (n≧3).

FIG. 5—Inhibition of TCR/CD3-mediated Ca²⁺-signalling of the human Jurkat T cell line by 8-Br-NHD.

Intact Jurkat T cells were loaded with Fura2-AM, and Ca²⁺ signalling was measured by digital ratiometric Ca²⁺ imaging (Guse et al., Nature 1999). For technical reasons the excitation wavelengths were set to 340 and 365 nm, whereas emission was filtered at 520 nm. The cells were preincubated with 250 or 500 μM 8-Br-NHD for 30 min. Then the cells were stimulated by anti-CD3 antibody OKT3 (10 mg/ml) in solution, and the cells were transferred to the examination chamber on the digital imaging station. The intracellular Ca⁺ concentration was then monitored for about 25 min with a sampling rate of 3 or 6/min. Data are presented as mean intracellular Ca²⁺ concentration ±SEM 25 min after stimulation. Numbers in brackets indicate the number of cells investigated in 3 independent experiments.

FIG. 6—Inhibition of proliferation of the human Jurkat T cell line by 8-Br-NHD. Proliferation of intact Jurkat T cells was measured as described earlier (Guse et al., Cell Calcium 1997). Various concentrations of 8-Br-NHD were added to the cultures in 96 well plates, and were present for 4 or 5 days. The number of living cells was determined every day by cell counting of trypan blue stained cells. Data are mean±SD from 2 independent experiments.

Materials and Methods

Synthesis of nicotinamide 8-bromohypoxanthine dinucleotide (8Br-NHD)

(a) Synthesis of 8-Bromo-inosine 5′-monophosphate

(b) Coupling with β-nicotinamide

Chromatography and Buffers

HPLC (Partisil) buffer: 50 mM KH₂PO₄, pH3, 5% HPLC Methanol (add methanol after adjusting the pH)

HPLC (reversed phase C18) buffer: 100 mM TEAA (triethylammonium acetate), pH5, 5% aqueous HPLC Methanol (1:1, MeOH: H₂O).

TEAB Buffer: Triethylamine (1M) in MQ water, bubble CO₂ in. Keep bubbling constant during the purification.

TEAA Buffer: Triethylamine (1M) and acetic acid (1M) in MQ water, pH 3.9.

TFA Buffer: Trifluoroacetic acid (150 mM) in MQ water.

HEPES Buffer: HEPES (25 mM) in MQ water, pH 7.3.

a) Synthesis of 8-Bromo-inosine 5′-monophosphate

Synthesis of 8-bromo-adenosine

Adenosine (3 g) was dissolved in DMF with heating (50° C.). N-Bromo succinimide (NBS) (2 g) was then added. The solution was kept under nitrogen and in the dark and stirred for 2 hr. More NBS (1 g) was added and the solution was stirred overnight under the same conditions. Some more NBS (1 g) was required to complete the reaction. DMF was removed under high vacuum. The residue was dissolved in methanol, and 20 g of silica were added. Methanol was removed under reduced pressure. The silica-pre-absorbed crude reaction mixture was loaded on a silica column pre-equilibrated with CHCl₃/MeOH/NH4OH. (100/4/1). 8-Bromo adenosine was isolated as the first fraction when eluted with CHCl₃/MeOH/NH₄OH (10/4/1). Some residual adenosine was isolated as the second fraction. 8-Br-adenosine was isolated in 75% yield as a yellowish solid (2.9 g).

Structure confirmed by NMR and TLC.

Synthesis of 8-bromo-adenosine monophosphate

8-Br-Adenosine (200 mg) was dissolved in hot triethyl phosphate (3 ml). The solution was gently heated with a heat gun under nitrogen. As soon as all the 8-Br-adenosine was dissolved, the solution was cooled to 0° C. Phosphorus oxychloride (0.1 ml) was then added dropwise to the solution. The reaction was stirred at 0° C. for 2 hr. The reaction was followed by HPLC (Anion exchange Partisil analytical column). More phosphorus oxychloride (0.05 mL) was added and the solution was stirred overnight at room temperature. As will be readily appreciated by those skilled in the art, these conditions may be altered depending on the dryness of the starting materials. Few precautions were taken to thoroughly dry 8-Br-adenosine. The solution was cooled to 0° C. and ice cold water (100 mL) was added. The mixture was stirred for 30 min at 0° C. and for 30 min at room temperature. Ethyl acetate (3×200 mL) was added to extract triethylphosphate from the aqueous layer. This latter was then concentrated to dryness and purified on a Q-Sepharose anion exchange column (height 15-18 cm, diam. 2.5 cm) using an isocratic gradient of TEAB (triethyl ammonium bicarbonate) buffer going from 0 to 1 M over 950 mL. 8-Bromo adenosine monophosphate was isolated as the last fraction eluted off the column at concentration of TEAB close to 1 M.

The structure was confirmed by NMR and HPLC.

Synthesis of 8-Br-inosine monophosphate

The pH of the solution containing the crude 8-Br-adenosine monophosphate (from 200 mg of adenosine) obtained after quenching of the phosphorylation step with ice cold water was brought to 3 by addition of few millilitres of 10N NaOH solution. Sodium nitrite (2.5 g) was then added. The solution was stirred for 5 hr, and 2.5 g of NaNO₂ were added. After an overnight stirring, the reaction mixture was concentrated under high vacuum to 20 mL, and loaded on a Norit activated charcoal column (height 10 cm, diam. 5 cm). The column was rinsed with 3 column volumes of water. Free phosphate and other salts were then eluted. 8-Bromo inosine and adenosine monophosphate were then eluted with 2 column volumes of a mixture of ethanol:H₂O:NaOH (90:9:1). To effect better elution of the nucleotides, the charcoal column may be poured into an Erlenmeyer (500 ml) in which a magnetic stirred was placed. A 200 ml mixture of ethanol:H₂O:NaOH (90:9:1) was then added and the solution was stirred for 30 min. The solution may then poured out. The same operation may be repeated twice (or until most of the nucleotides have come off the charcoal). The combined solutions were then filtered through Celite. The fractions were assayed by HPLC. The retention time difference between 8-Br-AMP and 8-Br-IMP is close to 1 min, with 8-Br-IMP eluting last. Combined fractions were concentrated. The contamination of 8-Br-IMP by 8-Br-AMP was negligible, and no further purification was necessary. Isolation of pure 8-Br-IMP from the Q-Sepharose anion exchange column was carried out by elution of the column with TFA (150 mM). However, the results varied greatly. Crude 8-Br-IMP (HPLC Partisil retention time: 3.4 min) was then used as it was for coupling to NMN.

b) Coupling with acetylated β-nicotinamide 5′-monophosphate

Synthesis of nicotinamide 8-bromohypoxanthine dinucleotide

1. Activation of 8-Br-IMP: 8-Br-IMP was dissolved in 3 ml of MQ water and added to a slurry of Dowex 50 (H⁺). The suspension was stirred for 30 min. The resin was filtered off and thoroughly rinsed with water. After concentration of the filtrate, methanol (10 ml) was added followed by 0.1 ml of trioctylamine. The solution was stirred for 30 min until a clear solution was obtained. Methanol was removed under vacuum, and the residue was concentrated three times from dry DMF (5 mL×3). After dissolving the residue in dry DMF (2 mL), dioxane was added (3 mL). Diphenyl phosphorochloridate (0.2 mL) was then added, immediately followed tributylamine (0.2 ml). The solution was stirred under N₂ for 4 hours. The solution became clear. The solvents were removed under high vacuum. Cold ether (20 ml×2) was then added to remove unreacted DPPC and amine.

2. Acylation of NMN: Nicotinamide mononucleotide (100 mg) was dissolved in 0.1 mL of MQ water and added to 10 ml of pyridine stirred on ice. Acetic anhydride (6 ml) was then added dropwise. The solution was stirred for 4 hours. The solvents were then removed under high vacuum. The temperature of the water bath was maintained below 30° C. Acetylated NMN decomposes rapidly otherwise. An aqueous solution (2 ml) of pyridine/water (1/9) was then added and the solution is stirred for 10 min at room temperature. After removal of the solvents, the residue was dissolved in DMF (5 ml×3), and the resulting solution was concentrated under high vacuum to remove any residual water.

3. Coupling of acetylated NMN and activated 8-Br-IMP: Acetylated NMN was resuspended in dry DMF (2 ml). A solution of activated 8-Br-IMP in dry DMF (3 mL) was then added, followed by dry pyridine (15 ml). The resulting solution was stirred for 24 hr under N₂. The flask containing the reaction mixture was covered with aluminium foil. The reaction was followed by ³¹P NMR, HPLC Partisil column: retention time of the acetylated 8-Br NHD was 4.9 min, while the precursors eluted at least 1 min faster. ¹H NMR (D₂0) δ, ppm: 9.44 (1H_{1Nic}, s), 9.28(1 H_{5Nic}, d, J=8.5 Hz), 9.00 (1H_{3Nic}, d, J=8.5 Hz), 8.34(1H_{4Nic}, dd, J=8.5, 8.5 Hz), 8.22 (1H_{2Ade}, s), 6.50 (1 H_{1Nic}, s), 6.07(1H_{1′Ade}, d, J=4 Hz), 5.50 (1H, s), 5.15(1H, dd, J=3, 3 Hz), 4.76-4.58 (2H, m), 4.51-4.20(4H, m), 3.16 (6_{HNCH2}, q, J=7 Hz), 1.25 (9_{HCH3}, t, J=7 HZ); ³¹P NMR (D₂0) δ, ppm: m, −0.11.7 to −12.4.

4. Upon completion of the reaction (major peak around −12 ppm), the solvents were removed, and HLPC grade methanol (10 mL) was added followed by 0.5 mL of concentrated aqueous ammonia. The solution was stirred at 0° C. for 6 hr. Methanol was then removed and the residue was dissolved in water. The pH was brought to 3 with 6M HCl, and the solution was loaded on a Q-Sepharose column (20 mm diam, 150 mm height) prewashed with 150 mM TFA (200 mL) and rinsed with 500 ml of MQ water. The column was rinsed with 200 mL of MQ water after the loading was completed. A gradient of TFA (0-100%) was used to elute 8-Br-NHD, an 80% isolated yield was obtained from 8-Br-AMP. The retention time of 8-Br-NHD using a Partisil analytical HPLC column is 3.2 min. 1H NMR (D₂0) δ, ppm: 9.40 (1H_{1Nic}, s), 9.21 (1H_{5Nic}, d, J=8.5 Hz), 8.95 (1H_{3Nic}, d, J=8.0 Hz), 8.31(1H_{4Nic}, dd, J=7.5, 7.5 Hz), 8.21 (1H_{2Ade}, s), 6.14 (1H_{1′Nic}, d, J=4.7 Hz), 6.03(1H_{1′Ade}, d, J=5.2 Hz), 5.16(1H, dd, J=5.5, 5.5 Hz), 4.80-4.10 (7H, m), 3.16 (6_{HNCH2}, q, J=7 Hz), 1.25 (9H_{CH3}, t, J=7 HZ); ³¹P NMR (D₂0) δ,ppm: −0.11.8 (d, J=20 Hz), −12.2 (d, J=20 Hz)

### Example 1

### Derivatives of NHD Inhibit Intracellular, Human ADP-Ribosyl Cyclases

Cyclic ADP-ribose (cADPR) has been discovered as a potent Ca²⁺-mobilising compound in sea urchin eggs (Lee, 1997). In the past decade, it has been shown that cADPR is also active in plants and in higher eukaryotes including a variety of mammalian tissues or cell types, such as cardiac and smooth muscle, pancreatic and parotid acinar cells, hepatocytes, PC12 cells GH₄C₁ cells, and T-lymphocytes (reviewed in Lee et al., 1997).

We have recently demonstrated in human Jurkat T-lymphocytes (i) that cADPR specifically releases Ca²⁺ from a D-myo-inositol 1,4,5-trisphosphate [Ins(1,4,5)P₃]-insensitive Ca²⁺ pool of permeabilized cells (Guse et al, 1995 and Guse et al, 1996), (ii) that cADPR stimulates sustained Ca²⁺ signalling in response to microinjection into intact cells (Guse et al, 1997), and (iii) that cADPR is an endogenous nucleotide (da Silva et al., 1998), and (iv) that cADPR is an essential second messenger in T cell Ca²⁺ signalling (Guse et al., Nature 1999).

As a potential link between TCR/CD3-stimulation and increased cADPR concentration, a novel ADP-ribosyl cyclase was detected recently at low basal activity in the cytosolic fraction of Jurkat cells (Guse et al., Nature 1999).

Here we demonstrate that the partially purified, soluble ADP-ribosyl cyclase from human Jurkat T cells is dose-dependently inhibited by 8-Br-NHD (FIG. 1) and also by NHD, 6-thio-NHD, and 7-deaza-NHD (FIG. 2). The NADase activity found in the same protein fraction was also inhibited with a similar pharmacology (FIGS. 1, 2).

A further intracellular ADP-ribosyl cyclase is the one found in heavy (P10) membranes from human Jurkat T cells. FIG. 3 shows that the intracellular, membrane bound P10 ADP-ribosyl cyclase was also dose-dependently inhibited by 8-Br-NHD. The NADase activity found in the same protein fraction was inhibited with a similar pharmacology (FIG. 3).

### Example 2

### Derivatives of NHD Inhibit a Cell Surface ADP-Ribosyl Cyclase

In addition to intracellular ADP-ribosyl cyclase, an ecto-ADP-ribosyl cyclase (CD38) is expressed in human T cells. Similar to the intracellular ADP-ribosyl cyclases, a dose-dependent inhibition of the ecto-ADP-ribosyl cyclase was observed with 8-Br-NHD (FIG. 4). The NADase activity found in the same protein fraction was inhibited with a similar pharmacology (FIG. 4).

### Example 3

### Derivatives of NHD Inhibit the Second Phase, Sustained Rise in Ca²⁺ Levels in Response to TCR/CD3 Stimulation

A sustained rise in Ca²⁺ levels in response to TCR/CD3 stimulation is an essential requirement for T lymphocyte proliferation. In addition, cADPR is an essential second messenger involved in the second, sustained phase of Ca²⁺ signaling.

In FIG. 5 it is shown that the ADP-ribosyl cyclase inhibitor 8-Br-NHD significantly reduced the second, sustained phase of Ca²⁺ signaling in intact human Jurkat T cells. The mechanism underlying this inhibition is most likely the inhibition of intracellular ADP-ribosyl cyclases, and the inability of the cells to produce cADPR in response to stimulation of the TCR/CR3 complex.

### Example 4

### Derivatives of NHD Inhibit Proliferation of Human Jurkat T Cells

In FIG. 6 it is shown that 8-Br-NHD dose-dependently inhibits proliferation of human Jurkat T cells, most likely by the inhibition of intracellular ADP-ribosyl cyclases.

Table 1 below shows the results of inhibition studies on the mitochondrial (P10) ADP-ribosyl cyclase from Jurkat T lymphocytes. It was shown that NHD, 8-Br-NHD, 7-deaza-NHD all inhibit P10 ADP-ribosyl cyclase and NADase. The compounds nicotinamide and β-NMN are published as inhibitors of NADase and were used as positive controls.

TABLE 1
Inhibition studies on the P10 ADP-ribosyl cyclase from Jurkat T
lymphocytes

Enzyme Activity (% of control) in the
presence of the Inhibitor (μM).

| | Cyclase activity | | | NAD glycohydrolase activity | | |
|---|---|---|---|---|---|---|
| Inhibitor | 10 | 100 | 1000 | 10 | 100 | 1000 |
| NHD | 63.6 | 11.26 | n.d | 59.0 | 0 | n.d. |
| 8-Br—NHD | 59.2 | 14.3 | n.d. | 60.8 | 15.6 | n.d |
| 7-deaza-NHD | 97.9 | 82.1 | n.d. | 97.5 | 83.3 | n.d. |
| Nicotinamide | 94.0 | 43.3 | 14.9 | 94.2 | 42.6 | 12.4 |
| β-NMN | 112.3 | 93.4 | 56.9 | 115.0 | 91.9 | 48.8 |

Table 2 below shows the results of inhibition studies on the soluble ADP-ribosyl cyclase from Jurkat T lymphocytes. It was shown that NHD, 8-Br-NHD, 7-deaza-NHD all inhibit P10 ADP-ribosyl cyclase and NADase. The compounds nicotinamide and P-NMN are published as inhibitors of NADase and were used as positive controls.

TABLE 2
Inhibition studies on the soluble ADP-ribosyl cyclase from Jurkat T
lymphocytes

Enzyme Activity (% of control) in the
presence of the Inhibitor (μM).

| | Cyclase activity | | | NAD glycohydrolase activity | | |
|---|---|---|---|---|---|---|
| Inhibitor | 10 | 100 | 1000 | 10 | 100 | 1000 |
| NHD | 46.0 | 10.6 | n.d | 37.3 | 13.5 | n.d. |
| 8-Br—NHD | 57.7 | 13.0 | n.d. | 55.5 | 3.3 | n.d |
| 7-deaza-NHD | 94.0 | 69.3 | n.d. | 94 | 80.5 | n.d. |
| Nicotinamide | 99.2 | 38.2 | 10.6 | 95.2 | 29.6 | 8.2 |
| β-NMN | 80.5 | 65.4 | 36.5 | 81.3 | 59.4 | 26.9 |

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in chemistry, biology or related fields are intended to be within the scope of the following claims.

### REFERENCES

Albrieux, M., H. C. Lee, and M. Villaz. 1998. J. Biol. Chem. 273:14566-14574.

Bezprozvanny, I., J. Watras, and B. E. Ehrlich. 1991 Nature 351:751-754.

Cancela, J. M., G. C. Churchill, and A. Galione. 1999. Nature (Lond.) 398:74-76.

Chini, E. N., K. W. Beers, and T. P. Dousa. 1995. J. Biol. Chem. 270:3216-3223.

Clapper, D. L., T. F. Walseth, P. J. Dargie, and H. C. Lee. 1987. J. Biol. Chem. 262:9561-9568.

Da Silva et al J. Chromatogr. B707 43-50 (1998).

Guse, A. H., Berg, I., da Silva, C. P., Potter, B.V.L., & Mayr, G. W., J. Biol. Chem. 272, 8546-8550 (1997).

Guse, A. H., Ca²⁺ signalling in T-lymphocytes, Crit. Rev. Immunol. 18, 419-448 (1998).

Guse, A. H., et al. Nature (Lond.) 398:70-73 (1999).

Guse, A. H. et al J. Immunol. 155 3353-3359 (1995).

Guse, A. H. et al J. Biol. Chem. 271 23946-23954 (1996).

Guse, A. H. et al Cell Calcium 22 91-97 (1997).

Jayaraman, T., Ondriasove, E., Ondrias, K., Harnick, D. J., & Marks, A. R., Proc. Natl. Acad. Sci. USA 92, 6007-6011 (1995).

Jenkins, M., D. M. Pardoll, J. Mizuguchi, T. M. Chused, and R. H. Schwartz. 1987. Proc. Natl. Acad. Sci. USA 84:5409-5413.

Lee, H. C., and R. Aarhus. 1995. J. Biol. Chem. 270:2152-2157.

Lee, H. C. et al Physiol. Rev. 77 1133-1164 (1997).

Murphy, C. T., A. M. Riley. S. J. Mills, C. J. Lindley, B.V.L. Potter, and J. Westwick. 2000. Mol. Pharmacol. 57:595-601

Rahavendran, S. V., and H. T. Karnes. 1993. Pharm. Res. 10:328-34.

1. The use of a compound of formula (Ia): 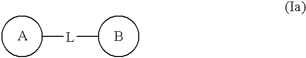
wherein A and B are independently selected from a cyclic ring, wherein each of which cyclic rings A and B may be optionally substituted at at least one ring position; and
L is a suitable linker;
or a pharmaceutically acceptable salt thereof, in the manufacture of a medicament for use in inhibiting ADP-ribosyl cyclase.

2. The use according to claim 1 wherein one or more of the cyclic rings A and B is a heterocyclic ring.

3. The use according to claim 1 or 2 wherein one or more of the cyclic rings A and B is a five membered ring.

4. The use according to any one of claims 1-3 wherein cyclic ring A has the formula (II): 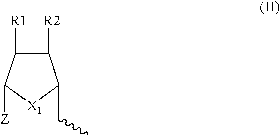
wherein X₁ is independently selected from O, S, CH₂ or a halo derivative thereof;
each of R₁ or R₂ is a substituent group independently selected from OH, OR, SH, SR, halo (preferably F), NH₂, NHR or 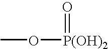
and wherein R is independently a hydrocarbyl group, preferably a C₁₋₁₂, preferably C₁₋₆, alkyl or acyl group (which may be optionally substituted), and
Z is a hydrocarbyl.

5. The use according to claim 4 wherein X₁ is O.

6. The use according to claim 4 wherein each of R₁ or R₂ is OH.

7. The use according to any one of the preceding claims wherein cyclic ring B has the formula (III): 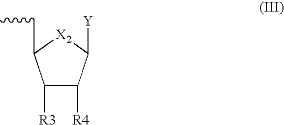
wherein X₂ is independently selected from O, S, CH₂ or a halo derivative thereof;
each of R₃ or R₄ is a substituent group independently selected from OH, OR, SH, SR, halo (preferably F), NH₂, NHR or 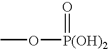
and wherein R is independently a hydrocarbyl group, preferably a C₁₋₁₂, preferably C₁₋₆, alkyl or acyl group (which may be optionally substituted); and
Y is a hydrocarbyl.

8. The use according to claim 7 wherein X₂ is O.

9. The use according to claim 7 wherein each of R₃ or R₄ is an OH.

10. The use according to any one of claims 4-9 wherein each of Y or Z is independently selected from an aromatic group or a substituted aromatic group

11. The use according to any one of claims 4-10 wherein each of Y or Z is independently selected from a heteroaromatic group or a substituted heteroaromatic group.

12. The use according to claim 11 wherein the heteroaromatic group or the substituted heteroaromatic group comprises a purine or a substituted purine structure.

13. The use according to claims 4-6 or to claims 10-12 when dependent upon claims 4-6 wherein Z is a pyridine or a substituted pyridine.

14. The use according to claims 4-6 or to claims 10-12 when dependent upon claims 4-6 wherein Z has the formula (IV): 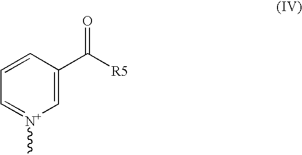
wherein R₅ is NH₂, OH or 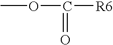
wherein R₆ is a hydrocarbyl group, preferably C₁₋₁₂, preferably C₁₋₆, alkyl or acyl group (which may be optionally substituted).

15. The use according to claims 7-12 wherein Y is a purine or a substitute purine.

16. The use according to claims 7-12 wherein Y comprises two fused heterocyclic rings, wherein each of said heterocyclic rings independently comprises nitrogen and carbon atoms in their respective rings, and wherein each of said heterocyclic rings may be optionally substituted at at least one ring position.

17. The use according to claim 16 wherein Y has the formula (V): 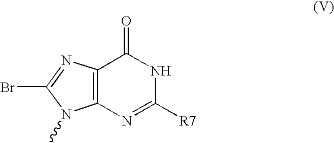
wherein R₇ is independently H or NH₂.

18. The use according to any one of the preceding claim wherein said linker is non-hydrolysable.

19. The use according to any one of the preceding claims wherein the linker has the formula (VI): 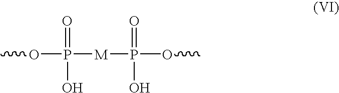
wherein M is independently selected from O, NH, CH₂ or a halo derivative thereof.

20. The use according to any one of the preceding claims wherein said compound is a compound of formulae (Ib): 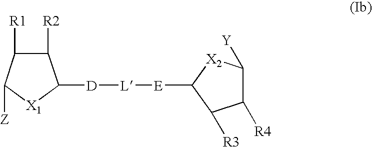
wherein D and E are independently selected from O, S, CH₂ or a halo derivative thereof;
wherein each of X₁ and X₂ is independently selected from O, S, CH₂ or a halo derivative thereof;
each of R₁, R₂, R₃ or R₄ is a substituent group independently selected from OH, OR, SH, SR, halo (preferably F), NH₂, NHR or 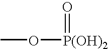
and wherein R is independently a hydrocarbyl group, preferably a C₁₋₁₂, preferably C₁₋₆, alkyl or acyl group (which may be optionally substituted);
each of Z and Y is a hydrocarbyl; and
L′ is the remainder of linker L;
or a pharmaceutically acceptable salt thereof.

21. The use of a compound according to any one of the preceding claims wherein said compound is one or more of a nicotinamide adenine dinucleotide analogue or a nicotinic acid adenine dinucleotide phosphate analogue.

22. The use of a compound according to any one of the preceding claims wherein said compound is one or more of: nicotinamide 8-bromohypoxanthine dinucleotide; nicotinamide 7-deazahypoxanthine dinucleotide; nicotinamide hypoxanthine dinucleotide; nicotinamide 6-thiohypoxanthine dinucleotide; nicotinamide 8-bromoguanine dinucleotide.

23. The use of a compound according to any one of preceding claims wherein said medicament is for use in modulating the immune response of a mammal.

24. The use of a compound according to any one of the preceding claims wherein said medicament is for use in treating an autoimmune disease or a graft rejection.

25. The use of a compound according to claim 24 wherein the autoimmune disease is selected from thyroiditis, insulitis, multiple sclerosis, iridocyclitis, uveitis, orchitis, hepatitis, Addison's disease, myasthenia gravis, rheumatoid arthritis and lupus erythematosus.

26. The use of a compound according to any one of the preceding claims wherein said medicament is for use in treating or preventing an immune disorder in a human or animal.

27. A pharmaceutical composition comprising a compound as defined in any one of the preceding claims or a pharmaceutically acceptable salt thereof admixed with a pharmaceutically acceptable carrier, diluent or excipient.

28. A pharmaceutical composition according to claim 27 wherein said composition comprises one or more additional pharmaceutically active compounds.

29. A compound of formula (Ib): 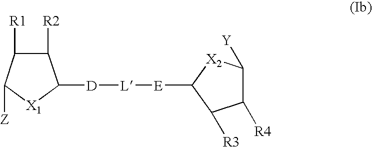
wherein D and E are independently selected from O, S, CH₂ or a halo derivative thereof;
wherein each of X₁ and X₂ is independently selected from O, S, CH₂ or a halo derivative thereof;
each of R₁, R₂, R₃ or R₄ is a substituent group independently selected from OH, OR, SH, SR, halo (preferably F), NH₂, NHR or 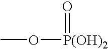
and wherein R is independently a hydrocarbyl group, preferably a C₁₋₁₂, preferably C₁₋₆, alkyl or acyl group (which may be optionally substituted);
each of Z and Y is a hydrocarbyl; and
L′ is the remainder of linker L.

30. A compound according to claim 29 wherein each of X₁ and X₂ is O.

31. A compound according to claims 29 or 30 wherein each of R₁, R₂, R₃ or R₄ is an OH.

32. A compound according to claims 29-31 wherein each of Y or Z is independently selected from an aromatic group or a substituted aromatic group

33. A compound according to claims 29-32 wherein each of Y or Z is independently selected from a heteroaromatic group or a substituted heteroaromatic group.

34. A compound according to claims 29-33 wherein the heteroaromatic group or the substituted heteroaromatic group comprises a purine or a substituted purine structure.

35. A compound according to claims 29-34 wherein Z is a pyridine or a substituted pyridine.

36. A compound according to claims 29-34 wherein Z has the formula (IV): 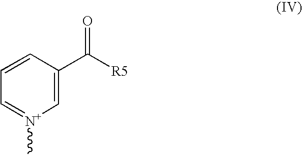
wherein R₅ is NH₂, OH or 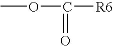
wherein R₆ is a hydrocarbyl group, preferably C₁₋₁₂, preferably C₁₋₆, alkyl or acyl group (which may be optionally substituted).

37. A compound according to claims 29-36 wherein Y is a purine or a substituted purine.

38. A compound according to claims 29-36 wherein Y comprises two fused heterocyclic rings, wherein each of said heterocyclic rings independently comprises nitrogen and carbon atoms in their respective rings, and wherein each of said heterocyclic rings may be optionally substituted at at least one ring position.

39. A compound according to claims 29-36 wherein Y has the formula (V): 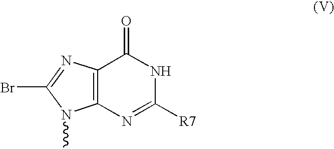
wherein R₇ is independently H or NH₂.

40. A compound according to any one of claims 29-39 wherein said linker is non-hydrolysable.

41. A compound according to any one of claims 29-40 wherein the linker has the formula (VI): 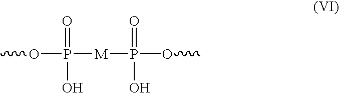
wherein M is independently selected from O, NH, CH₂ or a halo derivative thereof.

42. A compound according to any one of claims 29-41 wherein said compound is a nicotinamide adenine dinucleotide analogue.

43. A compound according to any one of claims 29-42 wherein said compound is one or more of: 8-bromo-nicotinamide hypoxanthine dinucleotide; 7-deaza-nicotinamide hypoxanthine dinucleotide; nicotinamide hypoxanthine dinucleotide; 6-thio-nicotinamide hypoxanthine dinucleotide.

44. A compound according to any one of claims 29-43 for use as a medicament.

45. Use of a compound according to any one of claims 29-44 in the manufacture of a medicament for use in inhibiting ADP-ribosyl cyclase.

46. A medicament comprising a compound according to any one of claims 29-45.

47. A method of inhibiting ADP-ribosyl cyclase comprising the step of contacting an ADP-ribosyl cyclase with a compound defined in any of claims 1-26 or 29-46 or a composition according to claims 27 or 28.

48. A method of modulating the concentration of cADPR and/or NAADP+in a cell comprising the step of contacting an ADP-ribosyl cyclase with a compound defined in any of claims 1-26 or 29-46 or a composition according to claims 27 or 28.

49. A method according to claim 48 wherein the concentration of cADPR is decreased.

50. A method according to claims 48 wherein the concentration of NAADP+is decreased to below an activating concentration, such as to a concentration less than or equal to 10 nM.

51. A method according to claims 48 wherein the concentration of NAADP+ is increased to an inactivating concentration, such as to a concentration greater than or equal to 10 μM.

52. A method of modulating intracellular Ca²⁺ levels in a T-cell comprising the step of contacting an ADP-ribosyl cyclase with a compound defined in any of claims 1-26 or 29-46 or a composition according to claims 27 or 28.

53. A method of modulating T cell activity, which comprises the step of contacting an ADP-ribosyl cyclase with a compound defined in any of claims 1-26 or 29-46 or a composition according to claims 27 or 28.

50. A method according to any one of claims 43-49 wherein said step is carried out in vitro.

51. A method according to any one of claims 43-49 wherein said step is carried out in vivo.

52. A method of treating or preventing a disease in a human or animal patient which method comprises administering to the patient an effective amount of a compound as defined in any one claims 1-24 or 27-40 or a composition according to claims 25 or 26.

53. A pharmaceutical pack comprising one or more compartments, wherein at least one compartment comprises one or more of the compounds defined in any of claims 1-24 or 27-40 or a composition according to claims 25 or 26.

54. A process of preparation of a pharmaceutical composition according to claims 25 or 26, said process comprising admixing one or more of the compounds defined in any of claims 1-24 or 27-40 with a pharmaceutically acceptable diluent, excipient or carrier.

55. An assay method for identifying an agent that can directly or indirectly inhibit ADP-ribosyl cyclase in order to treat an autoimmune disease or a graft rejection, the assay method comprising: contacting an agent with ADP-ribosyl cyclase; and measuring the activity of ADP-ribosyl cyclase; wherein a downregulation of activity of ADP-ribosyl cyclase in the presence of the agent is indicative that the agent may be useful in the treatment of an autoimmune disease or a graft rejection.

56. A process comprising the steps of:
(a) performing the assay according to claim 55;
(b) identifying one or more agents that can directly or indirectly downregulate the activity of ADP-ribosyl cyclase; and
(c) preparing a quantity of those one or more identified agents.

57. A method of treating an autoimmune disease or graft rejection, by down-regulating the activity of ADP-ribosyl cyclase in vivo with an agent; wherein the agent is capable of directly or indirectly downregulating the activity of ADP-ribosyl cyclase in an in vitro assay method; wherein the in vitro assay method is the assay method defined in claim 55.

58. Use of an agent in the preparation of a pharmaceutical composition for the treatment of an autoimmune response or a graft rejection, wherein the agent is capable of directly or indirectly downregulating the activity of ADP-ribosyl cyclase when assayed in vitro by the assay method according to claim 55.

59. An agent identified by the assay method according to claim 55.

60. An agent according to claim 59 for use in medicine.

61. An agent according to claim 60 for use in treating an autoimmune disease or a graft rejection.

62. Use of one or more compounds defined in any of claims 1-24 or 27-40 in an assay for identifying candidate compounds that are capable of influencing the activity of ADP-ribosyl cyclase.
